(19) United States
(12) Patent Application Publication
(10) Pub. No.: US 2023/0134521 A1
(43) Pub. Date: May 4, 2023

(54) COMPOUNDS FOR METHODS FOR PROMOTING FEMALE REPRODUCTIVE HEALTH THROUGH MEDIATION OF RENIN ANGIOTENSIN FUNCTION

(71) Applicant: Michael Emil Shanahan, Palisades, NY (US)

(72) Inventor: Michael Emil Shanahan, Palisades, NY (US)

(21) Appl. No.: 17/978,469

(22) Filed: Nov. 1, 2022

Related U.S. Application Data
(60) Provisional application No. 63/274,215, filed on Nov. 1, 2021.

(51) Int. Cl. A61K 31/085; A61K 31/565; A61K 45/06

(52) U.S. Cl. CPC A61K 31/085; A61K 31/565; A61K 45/06

(57) ABSTRACT

The invention provides safe and efficacious treatments for the improvement of sexual function such as Genitopelvic Pain/Penetration Disorders; vulvovaginal atrophy, vestibulodynia, dyspareunia, Sexual Interest/Arousal Disorder, low female libido and Female Orgasmic Disorder.

### PRIORITY CLAIM

This application claims priority to U.S. Provisional Application No. 63/274,215 filed Nov. 1, 2021 and is hereby incorporated by reference in its entirety.

### BACKGROUND OF THE INVENTION

It is widely recognized that the human body and its associated physiology are both complex and fragile in nature. Throughout a normal human lifespan, a person will suffer from various physiological “malfunctions” mostly in the form of aging and/or disease, which reduce the quality of life, and in certain severe circumstances, may result in death. Some of these conditions are gender specific or gender selective due to the various differences in male and female anatomy and physiology. For example, uterine, ovarian and cervical cancer are female specific, whereas conditions such as prostate and testicular maladies are male specific.

Masters and Johnson first characterized the female sexual response in 1966 as consisting of four successive phases: excitement, plateau, orgasmic, and resolution phases.

During sexual arousal, both the clitoris and the labia minora become engorged with blood and vaginal and clitoral length and diameter both increases. Masters and Johnson observed that the labia minora increase in diameter by two to three times during sexual excitement and consequently become everted, exposing their inner surface. In 1979, Kaplan proposed the aspect of ‘desire’, and the three-phase model, consisting of desire, arousal, and orgasm, with desire being the factor inciting the overall response cycle. This three-phase model is the basis for the DSM IV definitions of female sexual dysfunction, as well as the recent re-classification system made by the American Foundation of Urologic Disease (AFUD) Consensus Panel in October of 1998. Others have recently suggested that sexual function should be considered as a circuit, with four main domains: libido, arousal, orgasm, and satisfaction. Each aspect may overlap and/or negatively or positively feedback on the next.

The Sexual Function Health Council of the American Foundation convened the AFUD Consensus Panel, an interdisciplinary consensus conference panel, consisting of 19 experts in female sexual dysfunction selected from five countries. The panel included specialists for endocrinology, family medicine, gynecology, nursing, pharmacology, physiology, psychiatry, psychology, rehabilitation medicine, and urology. The objective of the panel was to evaluate and revise existing definitions and classifications of female sexual dysfunction. Specifically, medical risk factors and etiologies for female sexual dysfunction were incorporated with the pre-exciting psychologically based definitions 1998 AFUD Consensus Panel Classifications and Definitions of Female Sexual Dysfunction.

### Hypoactive Sexual Desire Disorder

The persistent or recurring deficiency (or absence) of sexual fantasies/thoughts, and/or receptivity to sexual activity that causes personal distress. Hypoactive Sexual Desire Disorder may result from psychological/emotional factors or be secondary to physiologic problems such as hormone deficiencies and medical or surgical interventions. Any disruption of the female hormonal milieu caused by natural menopause, surgically or medically induced menopause, or endocrine disorders, can result in inhibited sexual desire.

### Sexual Aversion Disorder

The persistent or recurring deficiency (or absence) of sexual fantasies/thoughts, and/or receptivity to sexual activity, that causes personal distress.

Sexual Aversion Disorder is generally a psychologically or emotionally based problem that can result for a variety of reasons such as physical or sexual abuse, or childhood trauma.

### Sexual Arousal Disorder

The persistent or recurring inability to attain, or maintain, adequate sexual excitement causing personal distress. It may be experienced as lack of subjective excitement or lack of genital (lubrication/swelling) or other somatic responses.

Disorders of arousal include, but are not limited to, lack of or diminished vaginal lubrication, decreased clitoral and labial sensation, decreased clitoral and labial engorgement or lack of vaginal smooth muscle relaxation. These conditions may occur secondary to psychological factors; however, often there is a medical/physiologic basis such as diminished vaginal/clitoral blood flow, prior pelvic trauma, pelvic surgery, or medications.

### Orgasmic Disorder

The persistent of recurrent difficulty, delay in, or absence of attaining orgasm following sufficient sexual stimulation and arousal that causes personal distress. This may be a primary (never achieved orgasm) or secondary condition, as a result of surgery, trauma, or hormone deficiencies. Primary anorgasmia can be secondary to emotional trauma or sexual abuse; however, medical/physical factors as well as medications (i.e., Seratonin re-uptake inhibitors) can contribute to or exacerbate the problem.

### Sexual Pain Disorders

Dyspareunia: recurrent or persistent genital pain associated with sexual intercourse. Dyspareunia can develop secondary to medical problems such as vestibulitis, vaginal atrophy, or vaginal infection. It can be either physiologically or psychologically based, or a combination of the two.

Vaginismus: recurrent or persistent involuntary spasms of the musculature of the outer third of the vagina that interferes with vaginal penetration, and which causes personal distress. Vaginismus usually develops as a conditioned response to painful penetration, or secondary to psychological/emotional factors.

Other sexual pain disorders: recurrent of persistent genital pain induced by noncoital sexual stimulation. This includes anatomic and inflammatory conditions including infections (ie HSV), vestibulitis, prior genital mutilation or trauma and endometriosis.

These classifications are subtyped as lifelong versus acquired, generalized versus situational, and organic versus psychogenic or mixed. The etiology of any of these disorders may be multifactorial and often times the disorders overlap.

Physiologic changes in the vagina during sexual arousal. During sexual arousal, genitals swell and expand as a result of increased blood flow.

Physiologic and biochemical mediators of the female sexual arousal response. Within the central nervous system, the medial preoptic, anterior hypothalamic region, and related libmic-hippocampal structures are responsible for sexual arousal. Upon activation, these centers transmit electrical signals through the parasympathetic and sympathetic nervous system. The neurogenic mechanisms modulating vaginal and clitoral smooth muscle tone, and vaginal and clitoral vascular smooth muscle relaxation are currently under investigation.

### Nonadrenergic/Noncholingeric Mediated Responses

Immunohistochemical studies in human vaginal tissues have shown the presence of nerve fibers containing NPY, VIP, NOS, CGRP. Preliminary studies suggest that vasoactive intestinal polypeptide (VIP) and nitric oxide (NO) are involved in modulating vaginal relaxation and secretory processes. In the clitoris, NO has been identified in human tissue and is believed to be a mediator of clitoral and labial engorgement. Organ bath analysis of rabbit clitoral cavernosal smooth muscle strips demonstrates enhanced relaxation in response to sodium nitroprusside and L-arginine (both NO donors), (Berman et al, unpublished data).

In organ bath studies, sildenafil causes dose-dependent relaxation of female rabbit clitoral and vaginal smooth muscle strips (unpublished observation) further suggesting a role for NO as a mediator of clitoral cavernosal and vaginal wall smooth muscle relaxation. However, the exact identity of the relaxatory NANC neurotransmitter remains unclear. VIP is a nonadrenergic/noncholinergic neurotransmitter that like NO, may play a role in enhancing vaginal blood flow, lubrication, and secretions. The vagina is heavily innervated with VIP-immunoreactive nerve fibers in close relation to the epithelium and blood vessels.14 In organ bath studies, VIP also causes dose-dependent relaxation of rabbit clitoral cavernosal and vaginal smooth muscle tissue, suggesting a similar role for endogenous VIP as a NANC neurotransmitter in clitoral and vaginal tissues (Berman et al, unpublished data).

### Impact of Hormones on Female Sexual Function

### Estrogen

Estrogen plays a significant role in regulating female sexual function. Estradiol levels affect cells throughout the peripheral and central nervous system and influence nerve transmission. A decline in serum estrogen levels results in thinning of vaginal mucosal epithelium and atrophy of vaginal wall smooth muscle. Decreased estrogen levels also result in a less acidic environment in the vaginal canal. This can ultimately lead to vaginal infections, urinary tract infections, and incontinence as well complaints of sexual dysfunction.15

Estrogens also have vaso-protective and vasodilatory effects which result in increased vaginal and clitoral and urethral arterial flow resulting in maintenance of the female sexual response by preventing atherosclerotic compromise to pelvic arteries and arterioles.16

With menopause, and the decline in circulating estrogen levels, a majority of women experience some degree of change in sexual function. Common sexual complaints include loss of desire, decreased frequency of sexual activity, painful intercourse, diminished sexual responsiveness, difficulty achieving orgasm, and decreased genital sensation. Masters and Johnson17 first published their findings of the physiologic changes occurring in menopausal women that related to sexual function in 1966. We have since learned that symptoms related to alterations in genital sensation and blood flow are, in part, secondary to declining estrogen levels, and that there is a direct correlation between the presence of sexual complaints and levels of estradiol below 50 pg/cm3.15

### Testosterone

Low testosterone levels are also associated with a decline in sexual arousal, genital sensation, libido, and orgasm. This can be accompanied by loss of pubic hair, vaginal mucosal thinning, and overall diminished sense of well being.13, 18 Therapeutic success with testosterone for inhibited desire in naturally menopausal women has been reported using a testosterone pellet.19 Testosterone also reportedly improves sexual desire in women, who are postmenopausal secondary to oophorectomy.20 There is evidence that testosterone supplementation decreases HDL levels and increases triglyceride levels, making women with history of hypercholesterolemia and cardiac disease at significant risk. Women with history of breast cancer should not be prescribed testosterone due to the fact that it can be converted to estrogen.

### Etiologies of Female Sexual Dysfunction

### Vasculogenic

The recently named clitoral and vaginal vascular insufficiency syndromes are directly related to diminished genital blood flow secondary to atherosclerosis of the iliohypogastric/pudendal arterial bed.21 Although other underlying conditions either psychological or physiological/organic may also manifest as decreased vaginal and clitoral engorgement, arterial insufficiency is one etiology that should be considered. Diminished pelvic blood flow secondary to aortoiliac or atherosclerotic disease leads to vaginal wall and clitoral smooth muscle fibrosis. This can ultimately result in symptoms of vaginal dryness and dyspareunia. Histomorphometric evaluation of clitoral erectile tissue from atherosclerotic animals demonstrates clitoral cavernosal artery wall thickening, loss of corporal smooth muscle and increase in collagen deposition.9 In human clitoral tissue, there is a similar loss of corporal smooth muscle with replacement by fibrous connective tissue in association with atherosclerosis of clitoral cavernosal arteries.9 While the precise mechanism is unknown, it is possible that the athersclerotic changes that occur in clitoral vascular and trabecular smooth muscle interfere with normal relaxation and dilation responses to sexual simulation.

Aside from atherosclerotic disease, alterations in circulating estrogen levels associated with menopause contribute to the age-associated changes in clitoral and vaginal smooth muscle. In addition, any traumatic injury to the iliohypogastric/pudendal arterial bed from pelvic fractures, blunt trauma, surgical disruption, or chronic perineal pressure from bicycle riding, for instance, can result in diminished vaginal and clitoral blood flow and complaints of sexual dysfunction.

### Neurogenic

The same neurogenic etiologies that cause erectile dysfunction in men can also cause sexual dysfunction in women. These include: (1) spinal cord injury or disease of the central or peripheral nervous system, including, diabetes and (2) complete upper motor neuron injuries affecting sacral spinal segments. Women with incomplete injuries retain that capacity for psychogenic arousal and vaginal lubrication.22 With regard to orgasm, women with spinal cord injury have significantly more difficulty achieving orgasm than normal controls. The effects of specific spinal cord injuries on female sexual response as well as the role for vasoactive pharmacotherapy in this population are being investigated. One recent report suggested a potential role for sildenafil in women with spinal cord injury.

### Hormonal/Endocrine

Dysfunction of the hypothalamic/pituitary axis, surgical or medical castration, menopause and premature ovarian failure, and chronic birth control use are the most common causes of hormonally based female sexual dysfunction. The most common complaints associated with decreased estrogen and/or testosterone levels are decreased desire and libido, vaginal dryness, and lack of sexual arousal. Estrogen improves the integrity of vaginal mucosal tissue and has beneficial effects on vaginal sensation, vasocongestion, and secretions, which all leads to enhanced arousal. Estrogen deprivation causes a significant decrease in clitoral intracavenosal blood flow and vaginal and urethral blood flow.3, 16 Histologically, diffuse clitoral fibrosis, thinned vaginal epithelial layers, and decreased vaginal submucosal vasculature. Thus, a decline in circulating estrogen levels can produce significant adverse effects on structure and function of the vaginal and clitoris, ultimately affecting sexual function. Androgen deficiency in women is characterized by impaired sexual function, lessened well-being, loss of energy, and negative effects on bone mass. Testosterone, together with its metabolite dihydrotestosterone, is the most potent endogenous androgen in both men and women. It is also the major precursor of estrogens. During the reproductive years, testosterone levels fall substantially, and by the mid-40s, circulating testosterone levels are approximately half of those of women in their 20s. There is, however, no dramatic decrease at the time of spontaneous menopause. A major fall does occur following bilateral ovariectomy.18

### Musculogenic

The pelvic floor muscles, in particular the levator ani and perineal membrane, participate in female sexual function and responsiveness. The perineal membrane, consisting of the bulbocavernosus and ischicavernosus muscles, when voluntarily contracted contributes to and intensifies sexual arousal and orgasm. In addition, they are responsible for the involuntary rhythmic contractions during orgasm. The levator ani muscles also modulate motor responses during orgasm as well as vaginal receptivity. When hypertonic, vaginismus can develop leading to or causing dyspareunia and other sexual pain disorders. When hypotonic, vaginal hypoanesthesia, coital anorgasmia as well as urinary incontinence during sexual intercourse or orgasm can develop.

### Psychogenic

In women, despite the presence or absence of organic disease, emotional and relational issues significantly affect sexual arousal. Issues such as self-esteem, body image, and the quality of the relationship with her partner can all affect her ability to respond sexually. In addition, depression and other psychological and mood disorders are associated with female sexual dysfunction. Furthermore, the medications commonly used to treat depressions can significantly affect the female sexual response. The most frequently used medications for uncomplicated depression are the serotonin re-uptake inhibitors (SSRIs). Women receiving these medications often complaint of decreased desire, decreased arousal, decreased genital sensation, and difficulty achieving orgasm. Several studies have recently been published documenting improvement in SSRI-induced sexual dysfunction in women with sildenafil.23

### Clinical Evaluation of the Female Sexual Response

Historically, evaluation of female patients with complaints of sexual dysfunction has been limited to psychological assessment. Physiologic evaluation of the female sexual response in the clinical setting has been complicated by the difficulty of objectively quantifying the changes that occur with arousal. Also, in contrast to the male erectile response, many genital changes that comprise the female sexual response are not only difficult to measure, but also may go unnoticed by the patient.

### Medical/Physiologic Evaluation

Evaluation of the patient with sexual dysfunction should contain a thorough physical examination including a pelvic examination, psychological and psychosocial assessment, laboratory or hormonal studies as indicated and physiologic monitoring of measures of arousal. In this way, both subjective and objective measures can be obtained and evaluated. The suggested hormonal profile includes FSH, LH, prolactin total and free testosterone levels, SHBG and estradiol levels. Testosterone is bound to both albumin and serum hormone binding globulin (SHBG) in the blood. SHBG levels increase with age and decrease with administration of exogenous estrogens.15, 16 If an abnormal estrogen and/or testosterone level is documented, appropriate replacement therapy can be initiated with subsequent resolution or improvement of the patient's symptoms. Women with low estrogen and/or testosterone levels typically experience symptoms of decreased libido, decreased sensation, vaginal dryness, dyspareunia, and decreased arousal.

Medical conditions including those that disrupt the hypothalamic—pituitary axis or hormone deficiencies secondary to menopause, chemotherapy, or following bilateral salpingo-oophorectomies should be identified.

Evaluating the female sexual response in the clinical setting, both validates the patient's problem and potentially diagnoses organic disease such as vascular insufficiency, hormonal abnormalities, or neurologic disorders. The studies being undertaken at our institution seek to define ranges of normal for the following parameters:

1) Genital blood flow: clitoral, labial, urethral, and vaginal peak systolic velocities and end diastolic velocities using Duplex Doppler Ultrasound.

2) Vaginal lubrication measurements.

3)Vaginal compliance/elasticity: pressure—volume changes.

4) Genital sensation: vibration perception thresholds as well as temperature perception thresholds.

These measurements can be recorded at baseline and following sexual stimulation. Eventually, definition of the parameters prior to initiating medical therapy may become the standard of care.

### Psychosocial/Psychosexual Assessment

In addition to the medical/physiologic evaluations, all patients should be evaluated for emotional and/or relational issues that may be contributing to her problem. This includes the context in which the patient experiences her sexuality, her self-esteem and body image, and her ability to communicate her sexual needs with her partner. This is an integral component of the female sexual function evaluation. Emotional and/or relational issues should be addressed prior to treatment, and certainly prior to determining treatment efficacy. If ongoing therapy is desired or required, it should also be provided.

One category of afflictions that specifically affect women is generally known as ‘Female Sexual Disorders.’ These disorders are defined in The Diagnostic and Statistical Manual of Mental Disorders (“DSM”), and include the following three categories: (1) Genitopelvic Pain/Penetration Disorder; (2) Sexual Interest/Arousal Disorder; and (3) Female

Orgasmic Disorder. All of these afflictions are common and known to significantly reduce the quality of life for millions of women and their sexual partners.

In the most current version of DSM, (DSM-V), older terminologies, both Hypoactive Sexual Desire Disorder (HSDD) and Female Arousal Disorder were merged into a single category of female sexual disorder now called Sexual Interest/Arousal Disorder (SIAD). Similarly, the formerly separate dyspareunia and vaginismus disorders are now collectively called Genitopelvic Pain/Penetration Disorder (GPPD). The terminology Female Orgasmic Disorder (FOD) remains unchanged.

GPPD is an underreported condition and can occur at any time in a woman's life cycle. 50% of postmenopausal women suffer from a degree of genital pain/penetration disorder, and 15% of all women will experience vestibulodynia, or provoked vulvar pain, at some point in their lifetime. The prevalence of sexual interest/arousal disorder (SIAD) varies, but studies indicate a range from about 26.7% of premenopausal women to about 52.4% of naturally post-menopausal women. Lastly, 4-10% of women do not have orgasms and up to 26% of women report having at least some difficulty with having an orgasm.

The DSM-V defines Genitopelvic Pain/Penetration Disorder, (GPPD) as difficulty in vaginal penetration, marked vulvovaginal or pelvic pain during penetration, or attempt at penetration (dyspareunia), fear or anxiety about pain in anticipation of, during, or after penetration, and tightening or tensing of pelvic floor muscles during attempted penetration (vaginismus).

For many women, however, pain may occur outside the context of penetration or sexual intercourse. For example, pain or discomfort may occur during any manipulation of their external genitalia (a condition known as vulvodynia). Such pain or discomfort may be due to vulvovaginal atrophy (VVA), including thinning of the vulvovaginal epithelium and a lack of lubrication and/or may be characterized as vulvodynia.

Vulvar pain is also a frequently occurring problem that affects both pre and post-menopausal women. Generally speaking, vulvodynia may present as more diffuse, constant vulvar pain, exhibiting a general sensation of burning or rawness in affected areas. Localized vulvodynia also known as vestibulodynia is pain around the opening of the vagina, the vulvar vestibule, most commonly found around the posterior hymenal ring between 4 and 8 o'clock, a region sometimes referred to by medical practitioners as the “posterior fourchette.” This is the most common cause of vulvar pain and the leading cause of dyspareunia in women under the age of 50.

This condition may manifest itself as primary vestibulodynia (i.e. pain with first attempted tampon and/or intercourse) or secondary vestibulodynia (i.e. development of vulvar pain following previously painless tampon use and/or intercourse). Conservative estimates indicate that at least 16% of women in the United States (˜14 million women) have experienced some form of chronic vulvar pain. This pain and discomfort seriously impacts and degrades the quality of life. Women suffering from this condition are in need of a clinically-proven, effective treatment, as they regularly self-treat symptoms, wasting both time and money on ineffective treatments. Vulvologists from around the world, including The International Society for the Study of Vulvovaginal Disease, and practitioners from multiple professions including: medicine, psychology, physical therapy and nursing have been unable to find an effective treatment for vestibulodynia and/or chronic vulvar pain, i.e., vulvodynia. To date, there has been no effective treatment for this life-altering vulvar pain.

Genitopelvic pain in general tends to be underdiagnosed because many women are either not asked about it in connection with regular medical exams or are uncomfortable discussing it with their healthcare professional. Furthermore, Genitopelvic Pain/Penetration Disorder (GPPD) frequently becomes the cause (or a significant contributing factor) in the manifestation of Sexual Interest/Arousal Disorder (SIAD) because the anticipation of pain during sexual encounters may, over time, form a negative cognitive response that inhibits sexual interest, desire and/or arousal.

One cause of Genitopelvic Pain/Penetration Disorder (GPPD) is vulvovaginal atrophy (VVA). Symptoms may include difficulty or inability to allow penetration, painful intercourse (dyspareunia), irritation, soreness, pain of the external genitalia, specifically of the vulva (vulvodynia), and dryness or decrease in lubrication from loss of mucous secretion. These symptoms exert a negative impact on the quality of life of up to 50% of all postmenopausal women.

Maturation index is a term used to indicate the percentage of vaginal cell types as determined from a cytological exam of epithelial cells from the upper one third of the vagina. It is typically expressed as percentages of each of three cell types (i.e., parabasal, intermediate and superficial cells) found on a wet prep exam. Premenopausal women with adequate estrogen levels have a maturation index of 40% to 70% intermediate cells, 30% to 60% superficial cells, and substantially no parabasal cells (<1%). In vaginal atrophy, there is an increase in the parabasal cell population and a corresponding decrease in superficial cells, evidence of the thinning of the vaginal epithelium.

Currently, there are, generally speaking, five types of treatments available to address the symptoms of vulvovaginal atrophy (VVA): 1) the topical or systemic use of exogenous estrogens, 2) The systemic use of a selective estrogen receptor modulator (“SERM”), dehydroepiandrosterone (DHEA), 3) carbon dioxide laser and, 4) the topical use of external lubricants or moisturizers. 5) More recently, an approach to treat such indications has been proposed by U.S. Pat. Nos. 9,750,716, 10,624,873, and 11,396,814 and its progeny to Epstein which rely on the topical only use of green tea catechins to improve sexual function (which are hereby incorporated by reference in their entirety including U.S. patent application Ser. No. 16/488,936 filed Feb. 23, 2018).

The lack of estrogen is believed to be responsible for at least some VVA symptoms in women. This estrogen deficiency may be due to age-related menopause, or iatrogenically induced menopause which is more likely the cause of estrogen deficiency in younger, pre-menopausal, women who are estrogen deficient. However, it is important to note that genital pain, involving both dyspareunia and vulvodynia, do occur in pre-menopausal women and/or in other women having essentially normal estrogen levels. Thus, not all Genitopelvic Pain/Penetration Disorders can be adequately addressed with exogenous estrogens.

Various forms of estrogens are currently available to treat women with, vulvovaginal atrophy, VVA, associated Genitopelvic Pain/Penetration Disorder, GPPD. They include oral estrogens with or without progestins, topical estrogens as creams, suppositories or solid hormone releasing rings (Estring). Bioidentical estrogens are 17 beta-estradiol, estrone, and estriol. Sources of estrogen maybe synthetic, animal source, (Premarin), or plant based extracted from soy or yams. DHEA is metabolized, in vivo, in peripheral tissues to both androgens and estrogens. DHEA causes proliferation of estrogen receptor-positive breast cancer cells. Regardless of the source, however, such compounds are all still estrogenic in their inherent effects, both beneficial and adverse, on estrogen sensitive target tissues.

As a result, many women either do not want or are unable to use oral or topical estrogens, which is the current standard therapy prescribed for treating symptoms of VVA. Despite being topical, estrogens applied locally to the vagina undesirably can and do cause statistically higher levels of plasma estradiol. There are significant risks to the use of both oral and topical estrogens in post-menopausal women with or without the adjunctive use of a progestin. The Women's Health Initiative (WHI) determined the following risks to be associated with estrogen use: 1) An increased risk of endometrial cancer in a woman with a uterus who uses unopposed estrogens; 2) An increased risk of stroke and deep venous thrombosis (“DVT”) in post-menopausal women using unopposed estrogens; 3) An increased risk of probable dementia in post-menopausal women using unopposed estrogens.

Further, a WHI study reported that women taking estrogen plus progestin had an increased risks of deep venous thrombosis (DVT), pulmonary embolism (PE), stroke and myocardial infarction (MI) in postmenopausal women. Additionally, an estrogen plus progestin ancillary study of the WHI reported an increased risk of developing probable dementia in postmenopausal women. The WHI estrogen plus progestin sub-study further demonstrated an increased risk of invasive breast cancer. Presently, there are no known safety studies to support the use of oral or topical vaginal estrogen in breast cancer survivors, and any estrogen use by breast cancer survivors is, considered to be, contraindicated by many health-care professionals.

Even while taking systemic (oral) estrogen, a significant number of women still suffer from certain residual VVA symptoms. Breast cancer treatment has been linked to an increase in the prevalence of VVA because the surgical, endocrine, and chemotherapeutic agents used in its treatment can cause or exacerbate VVA. As mentioned above, the use of locally applied estrogen treatment for this group of women remains controversial, and, at best, is only marginally effective.

Other currently available treatment options include the use of selective estrogen receptor modulators (SERMs), such Ospemifene (OSPHENA®), an FDA approved oral medication for moderate to severe dyspareunia (GPPD) associated with VVA due to menopause. Such SERMs act as a hormone by binding to the estrogen receptors ERa and ERβ, but bind more selectively to certain target organs such as the vagina, and thus produce less systemic estrogenic side effects compared to more non-selective oral estrogens. Ospemifene, however, exerts a strong, nearly full estrogen agonist effect in the vaginal epithelium as evidenced by improvement of the vaginal maturation index and decreased vaginal pH. It is an oral medication with both systemic and local estrogenic effects that has to be taken indefinitely to maintain its therapeutic effects. Oral Ospemifene carries the risk of increased cardiovascular events such as deep venous thrombosis, stroke, myocardial infarction, thickening of the endometrium, endometrial polyps, breast tenderness, and breast lumps.

Finally, treatment options for VVA include the use of vaginal moisturizers and lubricants to provide temporary relief from vaginal dryness and dyspareunia by reducing friction. Such compounds, however, do nothing to restore the normal anatomy or function of the female genitalia; thus, they have no long-term therapeutic effects. These exogenous topically applied intravaginal therapeutics can be messy as they will leak out of the vagina when a woman is in the upright position, and are not produced by a woman's body in response to normal physiological responses to sexual stimulation. This is also true for other topically applied treatments such as catechins. Vaginal estrogen therapy has proven more effective for VVA than all forms of non-hormonal therapy.

Another contributing cause of Genitopelvic Pain/Penetration Disorder (GPPD) is known as vestibulodynia, vulvodynia and/or vestibulitis. This affliction may be seen in up to 15% of all premenopausal women sometime in their lifetime. It is also a very common affliction of postmenopausal women that can be unrelated to VVA.

Female vestibulodynia may be generally described as a disorder of unknown etiology where there is localized provoked vulvar pain upon penetration of the vagina. There is also tenderness to touch around the vaginal opening (vestibule) during normal self or partner's manual sexual contact or during a health professional's physical examination. The entire area of the vestibule around the vaginal introitus can be affected but the experienced discomfort or pain is most commonly pronounced in the localized area of the posterior fourchette. The affected tissue in the vestibule has increased nerve endings and signs of inflammation and is typically painful. It occurs in women of all ages. It is estimated that approximately 15% of women (about 1 in 7) will experience this type of vulvar pain sometime in their lifetime.

Vestibulodynia may lead to dyspareunia or painful intercourse and thus interfere with sexual function by causing pain in genitalia. Vestibulodynia is a challenging disorder and to date there is no definitive treatment for this condition despite a range of treatments, which include: oral tricyclics, neuromodulators (i.e., gabapentin), topical anesthetics, botulinum injections, intralesional corticosteroid injections, biofeedback, sexual therapy, psychotherapy, physical therapy, and surgery as a last resort in an attempt to remove the painful tissue. Women who have vestibulodynia often simultaneously suffer from Sexual Interest/Arousal Disorder (SIAD) and Female Orgasmic Disorder (FOD) directly because of experiencing vestibulodynia, or provoked vulvar pain.

Sexual Interest/Arousal Disorder (“SIAD”) as specified in the DSM refers to “the persistent or recurrent inability to attain or to maintain sufficient sexual excitement, which causes personal distress.” In addition to absent or decreased sexual interest, including erotic thoughts or fantasies, there are four criteria that are taken into account to determine whether a woman suffers from SIAD. A woman has SIAD if she experiences personal distress caused by a decrease or lack of at least three of the following four criteria: 1) initiation of sexual activity or responsiveness to a partner's attempts to initiate it, 2) excitement and pleasure, 3) response to sexual cues, and 4) sensations during sexual activity, whether genital or non-genital. Again, three of the foregoing criteria are required for diagnosis. It may be expressed generally as lack of subjective excitement or lack of genital (lubrication/swelling) or other somatic responses.

The prevalence of Sexual Interest Arousal Disorder (SIAD), including low sexual desire, may range from about 26.7% of premenopausal women to 52.4% of naturally post-menopausal women. The disorder had no FDA-approved treatments (until August 2015 when Flibanserin, Addyi, was approved), and the FDA has recognized the condition as an area of unmet medical need.

Female orgasmic disorder, (FOD) as defined in the DSM, is the absence of orgasm (anorgasmia), infrequency or delay of orgasm, and/or reduced intensity of said orgasm. Such orgasmic dysfunction may also occur when a woman has difficulty reaching orgasm, even when sexually aroused with sufficient sexual stimulation. Many women have difficulty reaching orgasm with a partner, or during masturbation, even after ample sexual stimulation. Female Orgasmic Disorder (FOD) affects approximately one in three women.

It can be difficult to determine the particular underlying cause of Female Orgasmic Disorder (FOD). Women may have difficulty reaching orgasm due to one or more physical, emotional, and/or psychological factors. Contributing factors include: older age, medical conditions, such as diabetes, a history of gynecological surgeries, such as a hysterectomy, the use of certain medications, particularly selective serotonin reuptake inhibitors (SSRIs), mental health conditions, such as depression or anxiety, stress, societal negative stereotypes of women's sexuality, lack of adequate or effective sexual stimulation, etc. Sometimes, a combination of these factors can make achieving an orgasm difficult or not possible.

The inability to orgasm can lead to distress, which may make it even more difficult to achieve orgasm in the future. The main symptom of orgasmic disorder is the inability to achieve sexual climax. Women with Female Orgasmic Disorder (FOD) may have difficulty achieving orgasm during either sexual intercourse or during masturbation.

For many women, having unsatisfying orgasms, less intense orgasms, or taking longer than desirable to reach climax are common symptoms of FOD that lead to emotional distress.

The initial goal of therapy for Female Orgasmic Disorder is to enable the patient to reach an orgasm as desired under any circumstance. Underlying medical etiologies must first be considered including antidepressant-induced anorgasmia, anorgasmia secondary to substance abuse, and underlying neurological disorders.

Evidence about the effectiveness of psychological therapies in successfully treating FOD is inconclusive. Testosterone in combination with estrogen has been tried, but to date no pharmacologic agents have proved to demonstrate long-term beneficial effects on orgasmic function in women with FOD, beyond a placebo effect. A Canadian company is working on a low-dose nasal testosterone gel treatment for Female Orgasmic Disorder, FOD.

However, at present, no medication has been specifically approved by the US Food and Drug Administration (FDA) for the treatment of FOD. In addition, very little information is available about pharmacotherapy specifically targeting disorders of orgasm in women. The significant inherent risks of oral or topical hormonal therapy remain a concern.

Finally, over-the-counter (OTC) products (Zestra®) and nutritional supplements are marketed, but are ineffective in treating women with FOD. Thus, there is currently no safe or effective treatment for Female Orgasmic Disorder (FOD) that enables an orgasm or enhances the intensity of said orgasm. Therefore, for the treatment of Female Orgasmic Disorder (FOD), it is desirable to provide a therapeutic compound and associated treatment that is easy for the patient to use (e.g., a medication that can be applied to the affected area by the patient themselves) that shows significant improvement in orgasmic ease and intensity in a relatively short period of use and having no significant side-effects.

As discussed above, problem of SIAD encountered by many women reduces quality of life manifested as low female libido or sexual desire and decreased or absent sexual arousal. In the past, treatments for low female libido have had little success. Some physicians have prescribed various forms of off-label testosterone preparations to increase libido with limited results. Testosterone, however, is not approved by the FDA for treating libido problems in women. Long-term safety data on testosterone therapy for postmenopausal women who have a history of breast or uterine cancer, or who have cardiovascular or liver disease, is lacking, and is being studied. Available data suggests there may be an increased risk of heart attacks, stroke, hair loss on scalp, hair growth on face, acne, heart disease, clitoromegaly, risk of breast or uterine cancer through conversion of testosterone to estrogen, blood clots, deepening of voice, and fetal abnormalities (in pre-menopausal women).

Currently, there is no treatment for female SIAD. Thus, it desirable to provide a therapeutic compound and associated treatment that is easy for patients to use, that shows significant results in a relatively short period of time, and has no significant side-effects.

One attempt to create a safe and effective treatment for low female libido resulted in the development of the compound Flibanserin, by Sprout Pharmaceuticals, known by the tradename ADDYI, or more colloquially “The Pink Pill.”

Flibanserin, (BIMT 17 BS; 2H-benzimidazol-2-one, 1,3 -dihydro-1-[2-[4-[3(tri-fluoromethyl) phenyl]-1-piperazinyl]ethyl] is a non-hormonal, centrally acting molecule that acts as an agonist at postsynaptic 5-HT1A receptors and as an antagonist at 5-HT2A receptors

Approved by the FDA in August 2015, Flibanserin is the first and only drug indicated for pre-menopausal women for SIAD related disorders (formerly termed hypoactive sexual desire disorder). During FDA evaluation, Flibanserin, was twice rejected because according to FDA reviewers: “Flibanserin has a challenging benefit/risk assessment.” This is due to its relative lack of efficacy for the intended indication as well as the prevalence of certain undesirable side effects. For example, from a median baseline of about 2-3 satisfying sexual events (“SSEs”) per month, Flibanserin resulted in a median placebo-corrected increase of only about 0.5-1.0 SSEs per month over a six month period. Moreover, this marginal benefit was seen in only 18% of women taking 100 mg daily of the systemic drug for a minimum of 28 days.

Side effects of Flibanserin are significant and include syncope and somnolence. The risk of such side effects is amplified by drug interactions with CYP3A4 inhibitors such as oral contraceptives or Fluconazole and with concomitant alcohol intake. The FDA concluded that the clinically significant pharmacodynamic interaction between Flibanserin and alcohol is challenging to mitigate, particularly because Flibanserin requires daily use and alcohol consumption, including excessive drinking, is not uncommon in many cultures and further common for those engaging in sexual activity. The combination of ethanol and Flibanserin resulted in concerning cases of pre-syncope/syncope. In addition, Flibanserin trials were limited to generally healthy women who were not taking additional medication such as benzodiazepines, sleep aids, narcotics, or other medicines. Further, it is noteworthy that Flibanserin demonstrated no therapeutic effect at all on Genitopelvic Pain/Penetration Disorder (GPPD) including vestibulodynia and dyspareunia or on Female Orgasmic Disorder (FOD).

Furthermore, approximately 50% of all women who experience a form of sexual disfunction or FSD are hypertensive. See e.g, Female sexual dysfunction in essential hypertension: a common problem being uncovered by Michael Doumas et al. 2006 PMID: 17082720 DOI: 10.1097/01.hjh.0000251898.40002.5b. It is believed that such sexual dysfunction or FSD is either caused, contributed to, or exacerbated by hypertension. Although, there are many treatments for hypertension—some of which may provide some systemic relief—most are not well suited to adequately address the cause of some or all FSDs.

Thus, in view of the foregoing, there is currently a lack of safe efficacious treatments to improve overall sexual function in women as well as certain cutaneous inflammation disorders and treatments for Female Sexual Disorders.

It therefore would be desirable to provide compounds and methods of treatment for such female sexual disorders. In preferred embodiment, this would include oral administration of compounds for achieving this goal.

### SUMMARY OF THE INVENTION

The invention provides safe and efficacious treatments for Female Sexual Disorders, and or have a positive effect on libido, sexual performance, improve sexual sensation, as well as certain cutaneous inflammation associated with such dysfunction.

The invention provides a method for improving sexual function, comprising administration of a pharmaceutical composition including certain renin-angiotensin modulators that may be ingested orally or, in some embodiments, by applying the pharmaceutical composition to an individual's genital or erogenous area, wherein the pharmaceutical composition comprises a renin-angiotensin modulators or renin-angiotensin modulators analog.

In certain embodiments, the renin-angiotensin modulators may include Angiotensin-converting enzyme inhibitors (ACE inhibitors), Angiotensin II receptor blockers (ARBs), or renin inhibitors.

In some embodiments of the inventions described herein, said pharmaceutical composition further comprises a selective estrogen receptor modulator (SERM). In a preferred embodiment, said SERM is ospemifene. In other embodiments, said pharmaceutical composition further comprises certain amounts of estrogen. In other embodiments, said pharmaceutical compositions further comprise a combination of both a SERM and an estrogen hormone. In some embodiments, the pharmaceutical composition comprises a selective androgen receptor modulator (SARM). In some embodiments, the pharmaceutical composition comprises an androgen. In other embodiments, the pharmaceutical composition comprises a capsaicin. In some embodiments, the pharmaceutical composition includes a cannabinoid such as THC based compounds or CBD and/or may also include green tea based catechins such as EGCg which may be administered orally and/or topically.

In some embodiments of the invention, the pharmaceutical composition further comprises melatonin or Bremelanotide (PT-141) and/or hyaluronic acid in addition to the combinations described above.

The invention also provides methods and compositions for treating a male sexual disorder (MSD) or female sexual disorder (FSD), comprising orally administering a pharmaceutical composition, wherein the pharmaceutical composition comprises a renin-angiotensin modulator. In some embodiments, the pharmaceutical composition further includes a cannabinoid.

In other embodiments, the pharmaceutical composition further includes a selective estrogen receptor modulator (SERM) or a selective androgen receptor modulator (SARM). In other embodiments, the pharmaceutical composition further includes an estrogen, an androgen, or Capsaicin.

In some embodiments, A pharmaceutical composition for improving sexual function including libido is provided, comprising a renin-angiotensin modulator and a cannabinoid. In other embodiments, the pharmaceutical composition further includes a selective estrogen receptor modulator (SERM) and/or a selective androgen receptor modulator (SARM). In other embodiments, the pharmaceutical composition further includes an estrogen or an androgen. In other embodiments, the pharmaceutical composition further comprises a melatonin or Bremelanotide (PT-141).

In certain embodiments, the pharmaceutical composition may be delivered in a shampoo or body wash. In such embodiments, the shampoo or body wash may further comprise salicylic acid, selenium sulfide, or a suitable surfactant, wetting, adhesion and/or penetration agent.

In some embodiments, the pharmaceutical compositions of the invention further comprise an extract or essential oil from a source selected from the group consisting of ascorbic acid, vitamin E, omega 3, salt water, menthol, agar essential oil, agar extract, ajwain, aloe vera, amyris, angelica root, anise, balsam, basil, bay rum, bergamot, black pepper, buchu, butterbur, cajeput, cannabis flower, caraway, cardamom seed, carrot seed, cedarwood, Cedarleaf, chamomile, cinnamon, cistus, citrus vulgaris, citronella, clary sage, clove leaf, coriander, costmary, cranberry seed, cumin/black seed, cypress, davana, dill, eucalyptus, fennel seed, fenugreek, frankincense, galbanum, geranium, ginger, grapefruit, grape seed (e.g. Vitis vinifera), henna, jasmine, juniper berry, lavender, lemon, lemongrass, lime, litsea cubeba, lobelia/neem, melissa (Lemon balm), mentha arvensis/Mint, mugwort, mustard, myrrh, neroli, nutmeg, orange, oregano, orris, parsley, patchouli, perilla, pennyroyal, peppermint, pine, rose, rosehip, rosemary, rosewood, sage, sandalwood, sassafras, savory, schisandra, spearmint, star anise, tarragon, tea tree, thyme, vetiver, and yarrow ylang-ylang. In a preferred embodiment, the extract is from peppermint.

### DETAILED DESCRIPTION OF THE INVENTIONS

Aspects of the invention provide methods and compounds for treating female sexual disorders by modulating and/or regulating the renin-angiotensin cycle in afflicted individuals thereby improving both blood flow and restoring circulatory function in the reproductive organs, improving organ function and thereby minimizing or reducing pain and improving functional ability thus increasing neurological and physiological response to sexual stimuli.

Hypertension may generally be treated with numerous known classes of medications including, Diuretics, Beta-blockers, ACE inhibitors, Angiotensin II receptor blockers, Calcium channel blockers, Alpha blockers, Alpha-2 Receptor Agonists, Combined alpha and beta-blockers, Central agonists, Peripheral adrenergic inhibitors and vasodilators.

Diuretics help the body get rid of excess sodium (salt) and water and help control blood pressure. They are often used in combination with additional prescription therapies.

Beta-blockers reduce the heart rate, the heart's workload and the heart's output of blood, which lowers blood pressure.

ACE inhibitors. Angiotensin is a chemical that causes the arteries to become narrow (vasoconstrictor), especially in the kidneys but also throughout the body. ACE stands for Angiotensin-converting enzyme. ACE inhibitors help the body produce less angiotensin, which helps the blood vessels relax and open up (vasodilator), which, in turn, lowers blood pressure.

Angiotensin II receptor blockers. These compounds block the effects of angiotensin, a chemical that causes the arteries to become narrow. Angiotensin needs a receptor-like a chemical “slot” to fit into or bind with-in order to constrict the blood vessel. ARBs block the receptors, so the angiotensin fails to constrict the blood vessel. This means blood vessels stay open and blood flow is increased.

Calcium channel blockers. This drug prevents calcium from entering the smooth muscle cells of the heart and arteries. When calcium enters these cells, it causes a stronger and harder contraction, so by decreasing the calcium, the hearts' contraction is not as forceful. Calcium channel blockers relax and open up narrowed blood vessels, reduce heart rate and lower blood pressure.

Alpha blockers. These drugs reduce the arteries' resistance, relaxing the muscle tone of the vascular walls.

Alpha-2 Receptor Agonists. These drugs reduce blood pressure by decreasing the activity of the sympathetic (adrenaline-producing) portion of the involuntary nervous system. Methyldopa is considered a first line antihypertensive during pregnancy because adverse effects are infrequent for the pregnant woman or the developing fetus.

Central agonists. Central agonists also help decrease the blood vessels' ability to tense up or contract. The central agonists follow a different nerve pathway than the alpha and beta-blockers, but accomplish the same goal of blood pressure reduction.

Peripheral adrenergic inhibitors. These medications reduce blood pressure by blocking neurotransmitters in the brain. This blocks the smooth muscles from getting the “message” to constrict. These drugs are rarely used unless other medications don't help.

Blood vessel dilators (vasodilators). Blood vessel dilators, or vasodilators, can cause the muscle in the walls of the blood vessels (especially the arterioles) to relax, allowing the vessel to dilate (widen). This allows blood to flow through better.

Generally speaking, systemic blood pressure medications and vasodilators described above are known to have minimal if any effect on female sexual performance or health. Doses of Viagra and Levitra have been administered to women without improvement to sexual health, performance or libido and accordingly are not marketed as such. Similarly, other vasodilators or blood pressure medications such as diuretics, beta-blockers, calcium channel blockers, alpha blockers, alpha-2 receptor agonists, central agonists and Peripheral adrenergic inhibitors are also not known to if any material effect on female sexual performance or health and thus operate through a mechanism of action which as no direct effect on female sexual performance or health.

On the other hand, the renin-angiotensin (RA) cycle is one known metabolic system for controlling blood pressure and blood volume in humans, primarily through vasoconstriction, which has an impact female sexual health and wellbeing. One key component of that system is the angiotensin-converting enzyme (ACE) 2 which modulates the expression of ACE2 in host cells. ACE2, a pivotal component of the renin-angiotensin system, and exerts its physiological functions by modulating the levels of angiotensin II (Ang

II) and Ang-(1-7) in affected areas. The distribution and function of ACE2 in the female reproductive system shows that ACE2 is widely expressed in female reproductive organs including ovary, uterus, vagina, and vulvar regions. See for example, Potential influence of COVID-19/ACE2 on the female reproductive system by Yan Jin et al. Mol Hum Reprod. 2020 Jun. 1;26(6): 367-373.doi: 10.1093/molehr/gaaa030. PMID: 32365180. Therefore, such reproductive organs and tissues are significantly affected by angiotensin 2 which acts as vasoconstrictor restricts blood flow.

Regulating the RA cycle (e.g., through inhibition of the ACE2) affects blood flow in the female reproductive system including as well as modulating luteal angiogenesis and degeneration, which also influence changes in endometrial tissue. Generally speaking, improving blood flow by limiting the vasoconstriction caused by the RA cycle improves neurological function and facilitates production of a mucus membrane in the female reproductive organs and limits, minimizes or reduces endometriosis, a common cause of dyspareunia, as well as promoting normal lubrication and improved response to sexual stimuli. This is preferable and more efficacious to general systemic blood pressure reducers described herein which have no such targeted effect on female reproductive systems.

For example, during sexual arousal, improved genital engorgement occurs as a result of increased blood flow, which may have become have been reduced through certain AT-based vasoconstriction. With adequate local blood flow, the vaginal canal is lubricated by secretions from uterine glands and from a transudate that originates from the subepithelial vascular bed, passively transported through the intraepithelial spaces, sometimes referred to as intercellular channels. Engorgement of the vaginal wall raises pressure inside the capillaries and creates an increase in transudation of plasma through the vaginal epithelium. This vaginal lubricative plasma flows through the epithelium onto the surface of the vagina, initially forming droplets that coalesce to form a lubricative film that covers the vaginal wall. Additional moistening during intercourse comes from secretions of the paired greater vestibular or Bartholin's glands. In addition to lubricating, the vagina lengthens and dilates during sexual arousal as a result of relaxation of the vaginal wall smooth muscle. In human and animal models, sexual stimulation results in increased vaginal blood flow and decreased vaginal luminal pressure.

Moreover, physiologic changes occur in the clitoris and vestibular bulbs during sexual arousal. With sexual stimulation, increased blood flow to the clitoral cavernosal and labial arteries resulting in increased clitoral intracavernous pressure, tumescence, and protrusion of the glans clitoris, and eversion and engorgement of the labia minora. Studies show that, unlike the penis, the clitoris and vestibular bulbs lack a subalbugineal layer between the erectile tissue and the tunica albuginea layer. In the male, this layer possesses a rich venous plexus that, during sexual excitement, expands against the tunica albuginea, reducing venous outflow and making the penis rigid. The absence of this venous plexus in the clitoris and vestibular bulbs illustrates that this organ achieves tumescence, but not rigidity during sexual arousal, making increased blow and key aspect in sexual wellness and performance.

Accordingly, the present invention provides for the use of known AT cycle regulation compounds for vasodilation of affected erogenous and sexual reproductive zones in both male and females. Such compounds may include Angiotensin-converting enzyme inhibitors (ACE inhibitors) such as, but not limited to, Benazepril (Lotensin), Captopril (Capoten), Enalapril/Enalaprilat (Vasotec oral and injectable), Fosinopril (Monopril), Lisinopril (Zestril and Prinivil), Moexipril (Univasc), Perindopril (Aceon), Quinapril (Accupril), Ramipril (Altace), and Trandolapril (Mavik). In some embodiments, this may also include renin inhibitors such as Tekturna (Aliskiren) and/or may include Angiotensin II receptor blockers such as Azilsartan (Edarbi), Candesartan (Atacand) Eprosartan, Irbesartan (Avapro), Losartan (Cozaar), Olmesartan (Benicar), Telmisartan (Micardis), Valsartan (Diovan). The foregoing list is intended to be exemplary and other compounds with ACE inhibitor and/or ARB properties may be used if desired. Typically, only one of these compounds would be used. but multiple could be used for patients that demonstrate adequate response and tolerance to such combinations.

In accordance with aspects of the present invention, persons with known hypertension and female sexual dysfunction may be prescribed one of an ACE inhibitors, an Angiotensin II receptor blocker or renin inhibitor as described above to treat both indications. Dosages may begin a lowest therapeutic amount and increased accordingly as needed to effectively treat presenting symptoms, which may include sexual dysfunction, hypertension or both. In some embodiments, ACE inhibitor, an Angiotensin II receptor blockers may be preferred over renin inhibitors. Some embodiments may include hyaluronic acid to promote nerve regeneration and function and may include systemic vasodilators such as nitric oxide for fast acting kick off and well as other known vasodilators such as sildenafil, vardenafil etc. and the like.

In the case of non-hypertensive women with sexual dysfunction, such persons may be initially provided with treatment amounts below the minimum therapeutic dose recommended for hypertension treatment and then may be provided with increasing amounts to until undesirable low blood pressure results. For example, if one such AT cycle regulation compound has a minimum therapeutic dose of 200 mg-600 mg per day, an initial starting dosage for non-hypertensive could be half of the minimum hypertension amount, (e.g.,100 mg) may be provided and results monitored, and the dosage changed (increased or decreased) as treatment goals require. The therapeutic goal being to achieve treatment objectives with materially altering blood pressure. Prescribed doses may be increased or decreased accordingly to achieve treatment objectives. Furthermore, may not be necessary for such treatment to necessarily occur daily, but rather periodically (e.g., once, twice or three times a week) after an initialization stage where medication is provided daily until a steady state response is achieve and then tapering off as to an effective point for long term maintenance and treatment. The specific type of AT modulator and specific treatment regimen will be determined by a medical practitioner based on the patients physiological condition, response on other known factors influencing such as decision. As, above, some such embodiments may include hyaluronic acid to promote nerve regeneration and function and may include systemic vasodilators such as nitric oxide for fast acting kick off and well as other known vasodilators such as sildenafil, vardenafil etc. and the like.

As is known in the art, the therapeutic effects of pharmaceuticals on target tissues are frequently dose dependent. For virtually all drugs treatment protocols there are dosage parameters where the therapeutic benefit of the drug is manifest but where exceeding that therapeutic dosage range causes adverse, unwanted side effects, and in some cases, toxicity or exacerbation or worsening of condition for which the drug was supposed to benefit, which is generally undesirable and is therefore clinically avoided.

Sexual response includes desire, arousal, lubrication and orgasm. It is known that, if perceived as unpleasant or toxic, sensations of pain can interfere with a woman's sexual response. Genital pelvic pain disorder in VVA and vulvodynia, vestibulodynia is characterized by genital hypersensitivity and sensory hyperinnervation with unmyelinated sensory nociceptor neurons. Although direct effects of estrogen withdrawal on vaginal cells is implicated in VVA, (e.g., thinning of the vaginal epithelium) estrogen withdrawal also causes both autonomic and somatic sensory nerves to proliferate, suggesting that indirect effects mediated by changes in vulvar vaginal innervation may be a major contributor to pain associated with VVA. This has been shown by histopathological analysis of relevant tissues. Topical application of estradiol is a well-documented and effective therapeutic for pain associated with VVA.

Exogenous progesterone use, in contraceptives, in premenopausal women, may be one etiology of vulvar hyperinnervation in vestibulodynia and or vulvodynia. Progesterone can induce both autonomic and somatic sensory nerves to proliferate. Oral progesterones can increase the risk of developing vestibulodynia by four to ninefold. Topical estradiol has been shown to reduce the sensory hyperinnervation and vulvar pain associated with vestibulodynia and vulvodynia.

Symptoms of VVA and GPPD (vulvodynia, vestibulodynia and dyspareunia) can be caused by proliferation of autonomic and sensory nerve vulvar vaginal innervation density. Reduction or normalization of nociceptive (or pain afferent) pathways that are localized in the area of the vulva, vestibule, vaginal introitus, and posterior fourchette, in VVA, vulvodynia and vestibulodynia is the most likely mechanism for the therapeutic efficacy of the application of topical estrogens. Increased blood flow through the use of AT modulators increases vulvar mucosa and vaginal introitus and is effective in alleviating vulvar pain and dyspareunia in both VVA and vulvodynia/vestibulodynia.

Another mode of action of the present invention is on the improvement in mucous membranes which may help reduce inflammation and irritation that can be a factor in VVA, vulvodynia, and vestibulodynia. Increased sympathetic innervation may augment vasoconstriction and promote vaginal dryness.

The normal female sexual response depends on the function of the peripheral autonomic nervous system and intact signaling pathways controlling the tone of genital vascular and nonvascular smooth muscle.

The first measurable sign of sexual arousal is an increase in vaginal blood flow. This creates the engorged condition, that saturates the fluid resabsorptive capacity of the vaginal epithelium. This results in increase in vaginal fluid, i.e. lubrication which enables less friction during coitus.

Genital motor responses to sexual stimulation including vaginal lubrication is mediated by autonomic vasoactive neurotransmitters including, vasoactive intestinal peptide (VIP) and Nitric oxide synthetases.

The mechanisms underlying vaginal lubrication appear to be mediated by several vasoactive neurotransmitters especially Vasoactive Intestinal Peptide (VIP). VIP injection into or topical application to the vaginal wall increases vaginal blood flow and induces vaginal fluid production which may be mediated by AT modulators.

Nitric Oxide cyclic guanosine monophosphate pathway is involved in the physiological mechanism of female genital arousal. Endothelial Nitric Oxide Synthases (NOS) and VIP vasoactive intestinal peptides are co-localized in the human vagina. Nitric Oxide synthases are located on the vaginal vessels in close proximity to vasoactive intestinal peptides (VIP) on the vaginal nerve fibers. They are both directly involved in effecting vaginal blood flow and thus lubrication. Disturbances in these pathways are thought to contribute to the pathophysiology of sexual dysfunction. Nitric oxide relaxes genital vascular and nonvascular smooth muscle which plays a pivotal role in mediating the normal sexual response to visual and/or tactile erotic stimulation. Insufficient bioavailability of Nitric Oxide (NO) and/or vasoactive peptides decreases vasodilation which decreases vaginal lubrication. Proper use of AT modulators affects the level of NO in endothelial cells to increase lubrication.

An “effective amount” refers to an amount of therapeutic compound that is effective, at dosages and for periods of time necessary, to achieve the desired therapeutic or prophylactic result. A “therapeutically effective amount” of a therapeutic compound may vary according to factors such as the disease state, age, sex, and weight of the individual. A therapeutically effective amount may be measured, for example, by improved survival rate, more rapid recovery, or amelioration, improvement or elimination of symptoms, or other acceptable biomarkers or surrogate markers. A therapeutically effective amount is also one in which any toxic or detrimental effects of the therapeutic compound are outweighed by the therapeutically beneficial effects. A “prophylactically effective amount” refers to an amount of therapeutic compound that is effective, at dosages and for periods of time necessary, to achieve the desired prophylactic result. Typically, but not necessarily, since a prophylactic dose is used in subjects prior to or at an earlier stage of disease, the prophylactically effective amount will be less than the therapeutically effective amount.

An “individual,” “subject” or “patient” is a vertebrate. In certain embodiments, the vertebrate is a mammal. Mammals include, but are not limited to, primates (including human and non-human primates) and rodents (e.g., mice, hamsters, guinea pigs, and rats). In certain embodiments, a mammal is a human. A “control subject” refers to a healthy subject who has not been diagnosed as having a disease, dysfunction, or condition that has been identified in an individual, subject, or patient. A control subject does not suffer from any sign or symptom associated with the disease, dysfunction, or condition.

A “medicament” is an active drug that has been manufactured for the treatment of a disease, disorder, or condition.

“Patient response” or “response” can be assessed using any endpoint indicating a benefit to the patient, including, without limitation, (1) inhibition, to some extent, of disease progression, including stabilization, slowing down and complete arrest; (2) reduction in the number of disease episodes and/or symptoms; (3) inhibition (i.e., reduction, slowing down or complete stopping) of a disease cell infiltration into adjacent peripheral organs and/or tissues; (4) inhibition (i.e. reduction, slowing down or complete stopping) of disease spread; (5) decrease of an autoimmune condition; (6) favorable change in the expression of a biomarker associated with the disorder; (7) relief, to some extent, of one or more symptoms associated with a disorder; (8) increase in the length of disease-free presentation following treatment; or (9) decreased mortality at a given point of time following treatment.

As used herein, a “pharmaceutically acceptable carrier” or “therapeutic effective carrier” is aqueous or nonaqueous (solid), for example alcoholic or oleaginous, or a mixture thereof, and can contain a film-forming agent, adhesion agent, surfactant, emollient, lubricant, stabilizer, dye, perfume, preservative, acid or base for adjustment of pH, a solvent, emulsifier, gelling agent, moisturizer, stabilizer, wetting agent, time release agent, humectant, or other component commonly included in a particular form of pharmaceutical composition. Pharmaceutically acceptable carriers are well known in the art and include, for example, aqueous solutions such as water or physiologically buffered saline or other solvents or vehicles such as glycols, glycerol, and oils such as olive oil or injectable organic esters. A pharmaceutically acceptable carrier can contain physiologically acceptable compounds that act, for example, to stabilize or to increase the absorption of specific inhibitor, for example, carbohydrates, such as glucose, sucrose or dextrans, antioxidants such as ascorbic acid or glutathione, chelating agents, low molecular weight proteins or other stabilizers or excipients.

As used herein, “treatment” refers to clinical intervention in an attempt to alter the natural course of the individual or cell being treated and can be performed before or during the course of clinical pathology. Desirable effects of treatment include preventing the occurrence or recurrence of a disease or a condition or symptom thereof, alleviating a condition or symptom of the disease, diminishing any direct or indirect pathological consequences of the disease, decreasing the rate of disease progression, ameliorating or palliating the disease state, and achieving remission or improved prognosis. In some embodiments, methods and compositions of the invention are useful in attempts to delay development of a disease or disorder.

Cannabis and cannabinoid preparations have been linked to improvements of subjective indices of sexual function in women. These include self-reported increases in sexual desire, orgasmic function, sexual satisfaction, enjoyment, and pleasure. (Osborne & Fogfel, Substance Use & Misuse 43:539-572 (2008); Palamar et al., Arch Sex Behay. doi:10.1007/s10508-016-0782-7 (2016).) Furthermore, cannabinoids have been used to alleviate pain disorders. (Hill, Kevin, J. Am. Med. Assoc. 313(24):2474-2483 (2015).) AT-modulators can be combined with cannabinoids such as known THC or CBD based compounds treat Male and/or Female Sexual Disorders. The foregoing citations are incorporated by reference in their entirety.

### Other Observations

All publications and patent documents disclosed or referred to herein are incorporated by reference in their entirety. The foregoing description has been presented only for purposes of illustration and description. This description is not intended to limit the invention to the precise form disclosed. It is intended that the scope of the invention be defined by the claims appended hereto.

What is claimed is:

1. A composition for treating a female sexual disorder (FSD), the composition comprising an angiotensin modulator for oral administration, wherein said FSD is at least one selected from the group consisting of genitopelvic pain disorder (GPPD), vulvovaginal atrophy (VVA), vestibulodynia, dyspareunia, sexual interest arousal disorder (SIAD), and female orgasmic disorder (FOD).

2. The composition of claim 1, wherein the angiotensin modulator is selected from the group comprising Angiotensin-converting enzyme inhibitors (ACE inhibitors), Angiotensin II receptor blockers (ARB), or include renin inhibitors (RI).

3. The composition of claim 2, wherein the Angiotensin-converting enzyme inhibitors includes Benazepril (Lotensin), Captopril (Capoten), Enalapril/Enalaprilat (Vasotec oral and injectable), Fosinopril (Monopril), Lisinopril (Zestril and Prinivil), Moexipril (Univasc), Perindopril (Aceon), Quinapril (Accupril), Ramipril (Altace), and Trandolapril (Mavik), the renin inhibitor includes Tekturna (Aliskiren) and the Angiotensin II receptor blockers include Azilsartan (Edarbi), Candesartan (Atacand) Eprosartan, Irbesartan (Avapro), Losartan (Cozaar), Olmesartan (Benicar), Telmisartan (Micardis), Valsartan (Diovan).

4. The composition of claim 3, wherein said composition further comprises a selective estrogen receptor modulator (SERM).

5. The composition of claim 4, wherein said SERM is ospemifene.

6. The composition of claim 2, wherein said composition further comprises estrogen.

7. The composition of claim 3, wherein said composition further comprises a cannabinoid such as THC or CBD based compounds

8. The composition of claim 2, wherein said composition further comprises a melatonin or bremelanotide or hyaluronic acid.

9. A method for treating a female sexual disorder (FSD), comprising orally administering an angiotensin modulator, wherein said FSD is selected from at least one of the the group consisting of genitopelvic pain disorder (GPPD), vulvovaginal atrophy (VVA), vestibulodynia, dyspareunia, sexual interest arousal disorder (SIAD), and female orgasmic disorder (FOD).

10. The method of claim 9, wherein the angiotensin modulator is selected from the group comprising an Angiotensin-converting enzyme inhibitors (ACE inhibitors), Angiotensin II receptor blockers (ARB), or renin inhibitors (RI).

11. The method of claim 10, wherein the Angiotensin-converting enzyme inhibitors includes Benazepril (Lotensin), Captopril (Capoten), Enalapril/Enalaprilat (Vasotec oral and injectable), Fosinopril (Monopril), Lisinopril (Zestril and Prinivil), Moexipril (Univasc), Perindopril (Aceon), Quinapril (Accupril), Ramipril (Altace), and Trandolapril (Mavik), the renin inhibitor includes Tekturna (Aliskiren) and the Angiotensin II receptor blockers include Azilsartan (Edarbi), Candesartan (Atacand) Eprosartan, Irbesartan (Avapro), Losartan (Cozaar), Olmesartan (Benicar), Telmisartan (Micardis), Valsartan (Diovan).

12. The method of claim 9, wherein said composition further comprises a selective estrogen receptor modulator (SERM).

13. The method of claim 12, wherein said SERM is ospemifene.

14. The method of claim 10, wherein said composition further comprises estrogen.

15. The method of claim 10, wherein said composition further comprises a cannabinoid such as THC or CBD based compounds

16. The method of claim 10, wherein said composition further comprises a melatonin or bremelanotide.

17. The method of claim 10 wherein a dosage for the angiotensin modulator for non-hypertensive patients is less than the minimum therapeutic dose for treatment of hypertensive patients.

18. The method of claim 10 wherein the angiotensin modulator dosage is increased until a desired outcome is achieved without a substantially hypotensive result.

19. An article of manufacture for treating a Female Sexual Disorder (FSD) comprising (i) one or more dosages of a therapeutic composition including an angiotensin modulator component or a derivative thereof suitable for treating the FSD, and (ii) a label containing instructions for oral use in treating the FSD.

20. The article of manufacture of claim 19 wherein said angiotensin modulator component comprises one or more of the following: an Angiotensin-converting enzyme inhibitors (ACE inhibitors), Angiotensin II receptor blockers (ARB), or renin inhibitors (RI).

21. The article of manufacture of claim 20, wherein the Angiotensin-converting enzyme inhibitors includes Benazepril (Lotensin), Captopril (Capoten), Enalapril/Enalaprilat (Vasotec oral and injectable), Fosinopril (Monopril), Lisinopril (Zestril and Prinivil), Moexipril (Univasc), Perindopril (Aceon), Quinapril (Accupril), Ramipril (Altace), and Trandolapril (Mavik), the renin inhibitor includes Tekturna (Aliskiren) and the Angiotensin II receptor blockers include Azilsartan (Edarbi), Candesartan (Atacand) Eprosartan, Irbesartan (Avapro), Losartan (Cozaar), Olmesartan (Benicar), Telmisartan (Micardis), Valsartan (Diovan).
